(19) United States
(12) Patent Application Publication
(10) Pub. No.: US 2010/0106175 A1
(43) Pub. Date: Apr. 29, 2010

(54) METHOD AND DEVICE FOR GRAFT FENESTRATION

(76) Inventors: Criag Steven McLachlan, Westleigh (AU); Peter Mossop, East Melbourne (AU); Ian Nixon, Abbotsford (AU)

Correspondence Address: BAKER & DANIELS LLP, 300 NORTH MERIDIAN STREET, SUITE 2700, INDIANAPOLIS, IN, 46204, US

(21) Appl. No.: 12/523,598

(22) PCT Filed: Jan. 17, 2007
(86) PCT No.: PCT/AU07/00045

§ 371 Date: Jan. 4, 2010

(30) Foreign Application Priority Data
Jan. 17, 2006 (AU) 2006900222

(51) Int. Cl. A61B 17/34

(52) U.S. Cl. 606/185

(57) ABSTRACT

The present invention provides a method of creating fenestrations in situ through a body wall of a covered stent or endograft lumen. The fenestration is aligned with a side branch of the body lumen. The created fenestration of the graft is in communication with a side branch. That is the patent or open side branch permits fluid communication from the main lumen across the stented or endograft lumen. Tools are described to carry out these fenestrations for either a graft or a side branch that is communication with a graft in vivo. Further these tools are described to carry out these methods for both in situ branch tissue and graft fenestration and alignment of the fenestrations The present invention provides a method for in situ fenestration, the method including the steps of: positioning a graft or graft unit in situ within a body lumen and forming an initial void or space between graft unit and the inner wall of the body lumen. The present invention also provides a catheter including a: a) a distal tip that can perform a piercing and opening action; and or b) a distal tip rotates by means of a drive shaft within the catheter to cut and or open a tissue or graft to form fenestration; and or c) a stabilization means to stabilize the position of the catheter during fenestration.

### FIELD OF THE INVENTION

The present invention relates to in vivo (in situ) creation of fenestration of either a graft and or biocompatible material and or graft associated biological tissue. A method and device is described for fenestration that pertains to covered stents or endograft following their placement within a body lumen.

The fenestrations are created in situ through a body wall of a covered stent or endograft lumen. The fenestration is aligned with a side branch of the body lumen. The created fenestration of the graft is in communication with a side branch. That is the patent side branch permits fluid communication from the main lumen across the stented or endograft lumen. Tools are described to carry out these fenestrations for either a graft or a side branch that is communication with a graft in vivo. Further these tools are described to carry out these methods for both in situ branch tissue and graft fenestration and alignment of the fenestrations.

### BACKGROUND

Endovascular stent grafting has been successfully used in patients with abdominal and thoracic aneurysms and has been explored as a less invasive alternative in patients with stable type B aortic dissection. Furthermore, stent-graft placement has also been applied to treat aortic branch occlusions resulting from malperfusion syndromes in both type A and type B acute aortic dissections.

Patients with acute aortic dissection may have life threatening complications manifested by end organ ischemia. The mortality rate of patients with renal ischemia is 50% to 70% and as high as 87% in mesenteric ischemia. Although the surgical success rate at reversing peripheral pulse deficits is high, the surgical in-hospital mortality rates in the setting of end organ ischemia remain as high as 89%. As such, percutaneous management of this complication is therefore advantageous.

The prior art has included numerous endovascular grafts of varying design. In general, these endovascular grafts typically comprise: a tube of pliable material (e.g., expanded polytetrafluoroethylene (ePTFE) or woven polyester) in combination with a graft anchoring component (e.g., a stent, a frame, a series of wire rings, hooks, barbs, clips, staples, etc.) which operates to hold the tubular graft in its intended position within the blood vessel.

Depending on which region(s) of the aorta is/are involved, the aneurysm and or aortic dissection may extend into areas of bifurcation (i.e., the inferior end of the aorta where it bifurcates into the iliac arteries) or segments of the aorta from which smaller “branch” arteries extend. In this regard, the various types of aortic disease may be classified on the basis of the region(s) of involvement, as follows: 1) Thoracic Aorta—involving the aortic arch and branch arteries which emanate therefrom (i. e., the subclavian arteries); 2) Thoracoabdominal Aorta involving the descending thoracic aorta and branch arteries which emanate therefrom (i.e., thoracic intercostal arteries) and/or the suprarenal abdominal aorta and branch arteries which emanate therefrom (i.e., renal, superior mesenteric, celiac and/or intercostal arteries). 3) Abdominal Aorta involving the pararenal aorta and the branch arteries which emanate therefrom (i.e., the renal arteries) and 4) involving the infrarenal aorta with or without illiac involvement.

Unfortunately, not all patients diagnosed with aortic disease (in particular aneurysm and or dissection) are presently considered to be candidates for endovascular grafting. This is largely due to the fact that most endovascular grafting systems of the prior art are not designed for use in regions of the aorta from which side branches (i.e., carotid, innominate, subclavian, intercostal, superior mesenteric, celiac or renal arteries) extend. In fact aortic dissection usually occupies large distances that traverse many side branches and it is malperfusion of these side branches that characterizes the underlying pathophysiology for this condition.

In fact, most of the clinical experience with endoaortic grafting has been for the treatment of infrarenal aneurysms, with or without illiac involvement. Examples of endovascular grafting methods and systems useable to treat such infrarenal aneurysms include those described in the following U.S. Pat. No. 4,577,631 (Kreamer); U.S. Pat. No. 5,211,658 (Clouse); U.S. Pat. No. 5,219,355 (Parodi et al.); U.S. Pat. No. 5,316,023 (Palmaz et al.); U.S. Pat. No. 5,360,443 (Barone et al.); U.S. Pat. No. 5,425,765 (Tifenbrun et al.); U.S. Pat. No. 5,609,625; (Piplani et al.); U.S. Pat. No. 5,591,229 (Parodi et al.); U.S. Pat. No. 5,578,071 (Parodi); U.S. Pat. No. 5,571,173 (Parodi); U.S. Pat. No. 5,562,728 (Lazarus et al.); U.S. Pat. No. 5,562,726 (Chuter); U.S. Pat. No. 5,562,724 (Vorwerk et al.); U.S. Pat. No. 5,522,880 (Barone et al.); and U.S. Pat. No. 5,507,769 (Marin et al.).

The deployment of endovascular grafts within regions of the aorta from which branch arteries extend (e.g., regions of the aorta from which the renal, superior mesenteric, celiac, intercostal, and/or subclavian arteries emanate) present additional technical challenges because, in those cases, the endovascular graft must be designed, implanted and maintained in a manner which does not impair the flow of blood into the branch arteries or vessels.

Fenestrations have been added to pre-deployed custom-made tube grafts and commercially manufactured Zenith stent-graft systems to preserve perfusion of aortic side branches (J Endovasc Ther. 2001 February;8(1):16-24). However, the complexity of total alignment of these pre-made fenestrations still requires partial device deployment, orientation markers on the endograft, and complex intra-operative angiography enable maneuvering of the fenestration over the orifice of the target vessel with the aid of guiding catheters, however it does not take into account the precise geometry of each individual patient and the ability to correctly align the endograft in a period of time that does not cause significant ischemia or hypoxia to the targeted tissues. U.S. Pat. No. 5,425,765 (Tifenbrun et al.) has described an endovascular graft that has one or more pre-fabricated openings or pre-fabricated fenestrations formed at specific locations, to allow blood to flow from the aorta into one or more branch arteries. However, such fenestrations do not form discrete conduit(s) through which blood is channeled into each branch artery. As a result, the area surrounding the fenestrations could be prone to leakage of blood into the space between the outer surface of the aortic graft and the surrounding aortic wall especially if alignment is not exact. A further problem that always remains with each endograft procedure is the risk and need for potential bailout after an incorrect endograft landing where the graft covers a vessel side-branch; therefore methods that permit rapid in situ fenestration are necessary.

Additionally, fenestration of the endograft or a covered stent may not be the only requirement when aortic repair of a dissection is required, often tissue also has to be fenestrated to allow re-establishment of blood flow after a dissection flap is occluding a side branch. In these cases a pre-fenestrated endograft would not repair a tissue related problem. Therefore methods and tools to make exact fenestrations in situ are required.

Recently McWilliams et al (J Endovasc Ther. 2004 April;11(2):170-4) described the creation of fenestrations from a subclavian artery into a graft in order to reperfuse the occluded vessel covered by an endograft using a puncturing wire and follow-up dilatation of the graft entry point. Such a technique is similar to the puncture of an aortic dissection flap by a needle—the so called aortic fenestration procedure performed to improve flow within one or another lumen of the aorta following aortic dissection. Such a technique was first described by Williams et al in 1990—see Radiology. 1990 February;174(2):450-2).

The process of endograft fenestration described in the described invention differs in both method and device and represents a system of devices to address the problems of not only fenestrating the graft from within the graft lumen and into the adjacent vessel lumen but the also in a reverse fashion.

With regard the previous description by McWilliams et al (J Endovasc Ther. 2004 April;11(2):170-4) of endovascular fenestration: 1. There study has no specific fenestration device but simply refers to the off label use of a guide wire to puncture the graft and thereby result in an unpredictable and possible dangerous procedure; 2. Such graft entry via “off label” use of a guide wire could only be achieved from a branch vessel accessed peripherally via percutaneous retrograde access. This thereby limits the procedures applicability with mesenteric and renal vessels, which are not amenable to this approach.

Thus, in view of the above-discussed limitations and shortcomings, there remains a need in the art for the development of new endovascular grafting or covered stent systems and methods which permit in situ (in vivo) fenestration and this would be applicable to and have the following additive benefits where the following could be targeted a) endovascular grafting or covered stents in regions of a blood vessel (e.g., aorta) from which branch blood vessels (e.g., carotid, innominate, subclavian, intercostal, superior mesenteric, celiac, renal or iliac arteries) extend, and/or b) enable more aortic aneurysm and or aortic dissection patients to be considered as candidates for endovascular repair, and/or c) may otherwise advance the state of the art of endovascular grafting to improve patient outcomes or lessen complications, and/or d) permit bail out options for accidental occlusion of a side branch by an endograft and/or e) graft complex geometry that has been suggested to be too risky to date, such as the proximal ascending aorta and/or g) permit a non-staged procedure, that is the repair could be done during for example, one procedure instead of multiple procedures over time and or h) reduce operator time during the carrying out of these procedures.

Any reference herein to known prior art does not, unless the contrary indication appears, constitute an admission that such prior art is commonly known by those skilled in the art to which the invention relates, at the priority date of this application.

### SUMMARY OF THE INVENTION

The present invention provides a method for in situ fenestration, the method including the steps of:
positioning a graft or graft unit in situ within a body lumen and
forming an initial void or space between graft unit and the inner wall of the body lumen.

The void or space permits mechanical fenestration

The mechanical fenestration can be performed in the graft material.

The mechanical fenestration can be performed in a tissue associated with a side branch of the body lumen.

The mechanical fenestration can be performed in both the graft and tissue associated with the body lumen in which the graft is in situ.

The body lumen can be a large blood vessel

The body lumen can be an aorta

The void or space can be closed or removed after fenestration by the graft such that the graft has apposition with a luminal body wall.

The graft fenestration is aligned with a luminal body wall side branch

The graft is in a first open state to facilitate fenestration and then a final greater open state following fenestration.

The graft can be in a fully open state and held away from the luminal wall to create a void or space.

A catheter can be used to perform the steps of the method to create a void or space.

A conical or helical catheter can be used.

The legs on a catheter delivery system or graft can be used to perform the steps of the method of crating a void in which to create a fenestration safely.

After fenestration the graft can be in communication with inner lumen of the body lumen and side branches.

A fenestration can be created in both the graft and a side branch vessel.

There can be fluid communication between a side branch vessel and a graft fenestration.

The graft to be fenestrated can be an endograft

The graft to be fenestrated can be a covered stent

The graft to be fenestrated can be a biocompatible tube

The graft can be constructed from a woven or knitted biocompatible material.

The stent can be a stent frame made from a metal alloy

The stent can be covered or coated with a polymeric material.

The covering or coating can be a polyurethane polycarbonate.

The covering or coating can be selected from a plant derived material, polymer, hydrogel suitable for drug release

The fenestration can be carried out using one or more than one of the following: surgical laser, harmonic scalpel, guide wire, cutting device, needle, catheter, biopsy apparatus, ablation apparatus, ablation catheter, balloon catheter.

The void or space can allow for radiological assessment of side branches of a body lumen for example branches in communication with the aorta.

The radiological assessment of the side branches allows for the alignment of the graft fenestration with a side branch ostium.

The catheter is used to create a fenestration can include:
a) a distal tip that can perform a piercing and opening action; and or
b) a distal tip rotates by means of a drive shaft within the catheter to cut and or open a tissue or graft to form fenestration; and or
c) a stabilization means to stabilize the position of said catheter during fenestration. The catheter has a distal tip that is conical or helical or screw like to perform a piercing and opening action or a cutting and opening action. The distal tip can have a cutting type action.

The distal tip can be self-tapping.

The distal tip can be drawn into the graft and is self stabilizing

The distal tip or the catheter can be stabilized due to the act of fenestration and or completion of fenestration.

Stabilization of the distal tip or catheter can permit accurate alignment of the fenestration with a side branch body lumen and with the side branch vessel originating from a larger body lumen.

Stabilization of the distal tip or catheter can permit alignment of a side branch graft or stent with a side branch vessel in situ through an aortic graft and or stent.

Stabilization of the distal tip or catheter can occur prior to the act of fenestration via a stabilizing armature and or Balloon.

Stabilization of the catheter can occur prior to and during the act of fenestration.

Stabilization of the fenestration catheter can take place prior to fenestration via an external stabilizing armature and or balloon.

The armature or balloon can be part of the fenestration catheter system.

Fenestration can be made through a branch blood vessel and or an organ and or a aortic dissection flap and or a material related to a graft or stent device.

The catheter can be self tapping where by it can be drawn the tissue and is self stabilizing.

The present invention also provides a catheter including a:
a) a distal tip that can perform a piercing and opening action; and or
b) a distal tip rotates by means of a drive shaft within the catheter to cut and or open a tissue or graft to form fenestration; and or
c) a stabilization means to stabilize the position of said catheter during fenestration.

The distal tip can be conical, helical or screw like which rotates.

The distal tip can provide a cutting action.

The distal tip can be self-tapping.

The distal tip can be adapted to be drawn into the graft and can be self stabilizing

The distal tip or the catheter can be stabilized due to the act of fenestration and or completion of fenestration.

The distal tip or catheter can permit accurate alignment of the fenestration with a side branch body lumen and with a side branch vessel originating from a larger body lumen.

The stabilization of the distal tip or catheter can permit alignment of a side branch graft or stent with a side branch vessel in situ through an aortic graft and or stent.

Methods and tools are described for performing fenestrations in grafts, endograft systems and or stents and or in biological tissues in situ. Methods are described for implanting a vascular graft into a patient's aorta for repairing thoracic or abdominal aortic aneurysms and for making a fluid connection or anastomosis with the tributary vessels or side-branches of the aorta, such as the renal, hepatic and mesenteric arteries.

The present invention has multiple aspects and formats. In particular it concerns the delivery of an endograft via a delivery catheter system into a body lumen, preferably a large vascular body lumen such as the aorta. In one aspect of the invention is such that when the endograft is expanded in situ (within the body vascular lumen) there it is at least a first stable opened position such that there is a void (secondary lumen) between the endograft and the inner lumen of the grated vessel, permitting fluid within the said void. The void is located between the outer endograft lumen and the inner lumen of the blood vessel. The final open endograft position allows interaction between the outer graft lumen and the inner luminal wall. This interaction between the graft and the lumen in turn permits interaction with side branches that branch from the lumen of the vessel luminal wall. Created via in situ fenestrations within the endograft wall preferably in its first position these inter-communications between the graft-vessel wall and side branches ensue when the graft is in contact with the luminal wall.

The first position of the deployed endograft permits the use of methods and tools to create a fenestration to the graft such that the created fenestration is in contact with a side branch of the lumen where the graft resides. These tools and methods are used in situ. Both the tools and graft at all times are stabilized to prevent displacement of either the tools or endograft during fenestration and alignment with pre-existing side branches off the lumen of a blood vessel.

In one aspect of the invention a covered stent or endograft is deployed within a body cavity lumen such that it is sub-maximally opened to a first position. This first position permits a void or space between the interface of the graft and lumen body cavity of the aorta.

Where this void or space is created between an endograft and the luminal surface of a blood vessel this would permit blood flow within this void or space. The void or space between an endograft outer lumen and the inner lumen of the blood vessel would offer the following advantages:
1) The ability to permit continued blood flow with in this void or space,
2) as a result of permitted blood flow this would in turn permit the perfusion of side branches that may be otherwise be occluded by a fully deployed endograft,
3) The space between the endograft and large vessel lumen permits imaging of side branches that emanate from the inner lumen of the targeted blood vessel to be grafted,
4) the void or space allows for the injection of radiological contrast agents,
5) the void or space can be imaged and the side branches identified relative to the geometry and position of the endograft, the void or space permits fenestration of the graft such that the side branches luminal openings communicate directly with the fenestrations within the graft when the graft is then deployed to its maximal state and fixed in position within the aorta.

### BRIEF DESCRIPTION OF THE DRAWINGS

FIG. 1: diagrammatically depicts a graft 4 inside a blood vessel 1, 3 and a void 6 created between the graft 4 and the luminal wall of the blood vessel 11. A fenestration in the graft 8 is created within the void or space using a fenestration device 5 and the tip of the fenestration device 9 creating the fenestration in the graft;

FIG. 2: diagrammatically depicts a graft 20 inside the aorta 21 at the level of the renal arteries 24. A fenestration is created within the graft 20 at 29 and this is aligned with ostium of the renal artery 24;

FIG. 3: diagrammatically depicts a perfusion catheter 31 that is placed inside the aorta 37. The perfusion catheter extends beyond a deployed graft 33 and may be in either the true lumen or false lumen or both. The perfusion catheter will have a number of side holes 32 that permits radiological contrast to enter the luminal space 34;

FIG. 4A: diagrammatically depicts, in side view a fenestration catheter instrument or device 43 within a graft 41 located within the aorta 46. The fenestration catheter will be used to create a fenestration between the graft 41 and the target branch vessel 44;

FIG. 4B: diagrammatically depicts, in side view another fenestration catheter instrument or device similar to that of FIG. 4A with like parts being like numbered

FIG. 4C; diagrammatically depicts a top view of the fenestration catheter or instrument of FIG. 4B;

FIG. 5: diagrammatically depicts a distal end of a fenestration catheter used to create a fenestration by means of a screw-like tip 51. The catheter has flexible distal drive shaft 52 and this drive shaft 52 can be driven by a high torque proximal drive shaft 53;

FIG. 5A: diagrammatically depicts a distal end of a fenestration catheter similar to that of FIG. 5, with like parts being like numbered, where the tip is a of a conical shape without an external helical screw thread as in the catheter of FIG. 5; and

FIG. 6: illustrates the fenestration instrument of FIGS. 5 and 5A which has a catheter with a distal end that consists of tip 51 that is permitted to rotate and or pierce.

### DESCRIPTION OF THE PREFERRED EMBODIMENTS

The present invention relates generally to the field of the treatment of aortic disease and in particular to endoluminal aortic grafts, which allow accurate placement of a covered stent or tube in the aorta and a means to create fenestrations within the covered stent or tube and or surrounding tissue. In particular the covered stent is capable of being deployed and positioned anywhere within the aorta and there is a means to create fenestrations in situ in vivo. Fenestrations can be created within the graft or surrounding tissues. These fenestrations serve one of two purposes: firstly to prevent occlusion of side branches off the aorta by aortic tissue and or by the graft when it is fully deployed and secondly to reestablish blood flow such as in an aortic dissection that may complicate side branch blood flow by means of a dissection flap.

A graft that is placed in the aorta or other body lumen can be any of the following or combinations thereof: a stent that are covered or partially covered, a non coated or covered stent, a stent that is completely enclosed and made from a metal alloy, a endograft that is a tube made of bio-compatible material such as woven or knitted materials, a tube that is formed from bio-compatible blood contacting polymer materials, a tube that is made from a coated polymer mesh, a tube that is a hybrid of these. The biocompatible material tube can in one embodiment be a DACRON material tube or a coated mesh.

A fenestration is a hole that is created within a graft or tissue. As used herein, fenestration means an opening having rectilinear sides such as rectangular, triangular or the like, or having curvilinear sides such as circular, oval or the like.

Methods to create a fenestration include but not limited to the use of lasers, guide wires, catheter ablation instruments, cutting tools, needles, and biopsy instruments.

There may be two or more in situ fenestrations created according to the number of intersecting arteries.

The fenestrations can be created within a graft covering only.

Alternatively the fenestrations are created in both tissue interacting with the graft and the graft.

Alternatively the fenestrations are created in tissue.

Fenestrations can be created from within the graft or outside the graft for example, within a void or space that exits between the graft and luminal wall of the blood vessel. The fenestration may also be created both within and outside the graft using tools that work on both sides of a graft wall to create a fenestration.

It is a preferment that, during the creation of fenestration of graft and or tissue, the graft is sub-maximally opened within an in situ position within the body lumen.

The graft can be opened sub-maximally in situ for example, as a result of its delivery system that permits the graft to open sub-maximally. In this case the catheter delivery system is restraining a maximal open state of the endograft. This restraint creates a potential for void or space that encompasses the delivery system. There is potential for the catheter delivery device and or catheter-restraining device to permit blood flow.

The void or space could therefore be potentially created via a catheter.

In a further embodiment the catheter delivery system has an outside rail that permits a retractable helical catheter to be positioned to create a void or space between the delivery system and or graft and the inner lumen of the aorta.

In another embodiment the delivery catheter is helical and can be used to position the graft and also create a void or space when the delivery system is flush or approximated to the aortic luminal wall.

The void or space created by a helical catheter can be due to a non-solid helical network. For example the helical catheter delivery system can be a mesh permitting blood flow across its helical structures. The helical catheter can also be like a large stent itself. Alternatively the delivery catheter has retractable legs that can be used to anchor the delivery system off the inner wall of the aortic lumen. The retractable legs may be flexible like a guide wire and or balloon type structures with voids that allow communicating blood flow within the created void or space along the length of the delivery catheter or graft.

In another embodiment the fenestrations are created in a graft that is maximally opened, yet the graft is prevented from interacting with the wall of the lumen and a void or spaces created between the outer lumen of the graft and the inner wall of the aortic blood vessel.

An interface void that exists between the graft and the luminal body wall permits mechanical fenestration of the endograft. Fenestration tools can be placed in the void or spacer within the graft to create the fenestrations.

If desired a fenestration catheter or guide wire can be used as a cutting tool for generating a fenestration on-demand between a covered graft within a body aortic lumen closely associated with a side blood vessel. The fenestration catheter is suitable for percutaneous introduction into and extension through a graft material.

The fenestration catheter described above can include:
a a tube having a fixed axial length, a discrete proximal end, a discrete distal end, and at least one internal lumen of predetermined volume;
b a distal end tip adapted for intravascular guidance of said tube through a endograft wall and forming a fenestration. The fenestration in the graft in association with a side branch blood vessel in-vivo;
c a means of anchoring the fenestration catheter positioned at said discrete distal end such that it is in a stationary position for axial alignment with the graft fenestration to be created and the said associated side branch blood vessel for which it will be in communication.

In another embodiment a fenestration catheter is used as a cutting tool for generating a fenestration on-demand in both a graft and side branch vessels separated at a chosen anatomic site by a void or space. The fenestration catheter being suitable for percutaneous introduction into and extension through a void or space to fenestrate the ostium of a blood vessel and to fenestrate the graft. in this instance the fenestrate catheter includes:
a a tube having a fixed axial length, a discrete proximal end, a discrete distal end, and at least one internal lumen of predetermined volume;
b a distal end tip adapted for intravascular guidance of said tube through a endograft and forming an association with a blood vessel in-vivo to a chosen anatomic site;
c a means of anchoring the fenestration catheter positioned at said discrete distal end such that it is in a stationary position for axial alignment outside the graft to create a fenestration within the graft and a fenestration with in an associated side branch blood vessel; and
d The created fenestration of the blood vessel and the graft fenestration are in communication such that when the void or space is closed and the graft also interacts with the lumen of the blood vessel and the graft outer wall.

The fenestration catheter can be anchored via legs from the fenestration catheter within the graft.

In another embodiment the fenestration catheter can be anchored within the void or space. Anchoring by legs in the void or space can include the following anchoring or attaching positions for the legs: the outer luminal vascular wall, the outer graft wall the catheter delivery system and or combinations thereof.

In another embodiment the fenestration catheter is anchored via these legs stabilizing themselves between the graft and the side branch.

In another embodiment the fenestration catheter is stationary and anchored via magnets.

In another embodiment the fenestration is created within the vessel-graft using a device specifically designed as a fenestration catheter and or tool and or instrument composed of a highly torquable shaft terminating in a flexible shaft segment joining a self-tapping screw tip mechanism rather than a cutting, puncturing or other traumatic method of graft fenestration previously described. The catheter and its tip are rotated by hand or by a low speed motor driver to initiate and expand the fenestration and reversal of rotation direction allows for atraumatic removal of the screw tip.

As the device is self tapping it is drawn into the fabric of the graft and is self stabilizing once activated in comparison to a technique which requires significant forward pressure to disrupt the graft fibers.

Once a fenestration has been created the device is removed and a guide wire is passed through the stabilizing guiding catheter which remains held in position at the point of fenestration. The created fenestration can be further increased in size as required by balloon angioplasty and a covered stent or endograft that is individual component and or is a component side branch of a stent or graft is then deployed into the side branch of a vessel to complete the vessel reconstruction.

In a further embodiment the fenestration catheter has a central lumen though either the shaft and or tip of the device which allows the passage of a guide wire directly into the target branch once crossing of the fabric has been achieved by the tip of the device.

The inventor has determined this method of using a fenestration catheter with a screw tipped device to be less traumatic in nature as it forms a smooth expansion of the graft material displacing graft fibers rather than disrupting them.

Subsequent expansion of the fenestration formed in this way is therefore less likely to create tearing of the graft with loss of graft integrity. This will result in a more robust entry for the implantation site for a side branch covered stent or branch endograft and reduce the possibility of leakage at the junction site.

In another embodiment there is provided a novel guiding catheter that delivers the fenestration device described and has a unique ability to stabilize and orient the fenestration catheter.

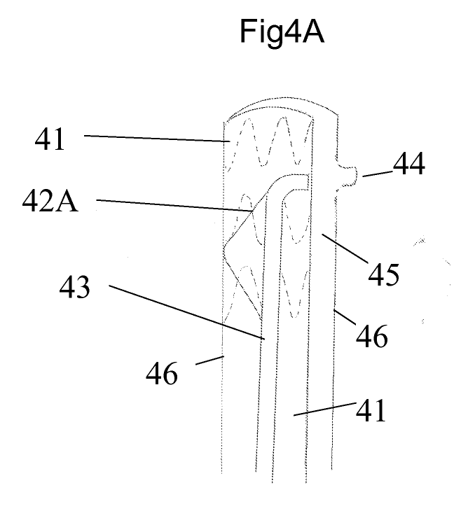

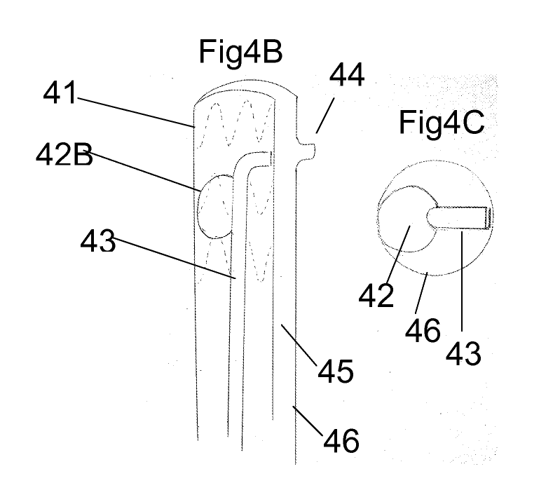

Such a device combines a pre-curved guiding catheter, which is locked into position through the use of a deployable armature or leg or deflection wire, which is of variable flexibility and is integrated with the catheter shaft. In another version the guiding catheter can be stabilized in position by an eccentric balloon. In all forms the guide system is braced within the endograft to allow the co-axial delivery of the fenestration catheter to its target, with the tip of the guide being pushed on to the endograft by the counter pressure of the stabilizing elements. The device is also described and depicted in FIGS. 4,5 and 6.

In another variation the guide catheter can have an integrated imaging system to visualize the target branched vessel—this can be ultrasound, angiography, real time MRI or other radiographic means but is not limited to these modalities.

Targeting of the branch can also be based on fluoroscopy using the presence of a previously implanted stent at the origin of the target vessel or can be achieved by the use of a novel infusion catheter designed to be placed between the graft and branch to allow perfusion of the branch vessel following deployment of the graft and to allow contrast opacification of the vessel for fluoroscopic guidance.

This device and method allows creation of fenestrations from both the internal or external aspect of the graft, for example, from the luminal aspect into the branch vessel or from the branch into the graft making it a generally applicable method for graft branch vessel formation. And in terms of creating graft fenestrations for connecting graft associated branching vessels the device is therefore a method for in situ in vivo endograft customization.

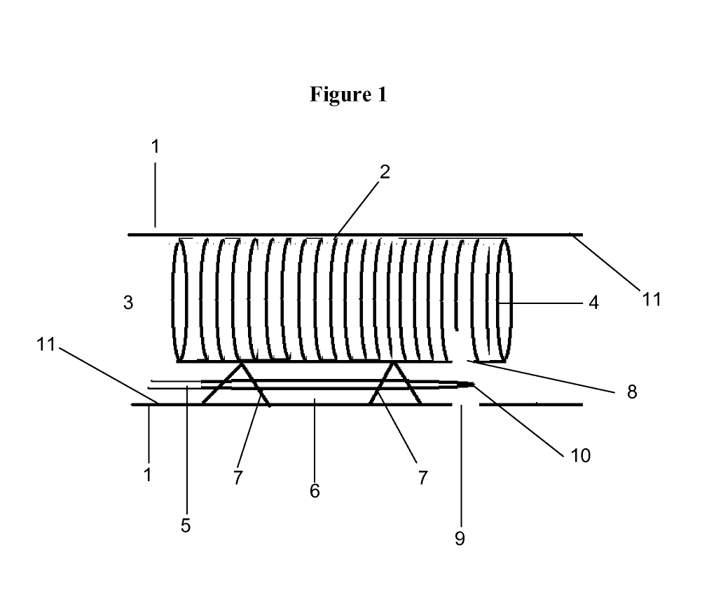

Illustrated in FIG. 1 is a graft 4 is depicted in situ within a large blood vessel 3. The outer wall of the vessel is defined 1 and the inner luminal wall of the blood vessel is also defined 11. There is a void or space 6 created between the graft 4 and the inner wall of the blood vessel 11. The void or space is created in this situation by retractable legs 7 arising from the graft 4. The graft legs position the graft between the luminal wall 11 of the blood vessel. Blood flow can move through the lumen of the blood vessel 3 and through the graft 4 and through the void or space 6. Also through the void or space a guide wire 5 can be delivered to perform at its tip 10 a fenestration 8 in the graft. The fenestration is created in the graft 4 at a position that is directly opposite to the ostium 9 of a side branch vessel. When the void or space 6 is removed and the graft 4 and the side branch ostium 9 will be in communication. The top of the cross section shows that the delivery system 2 in association with the graft 4 does not permit a void or space.

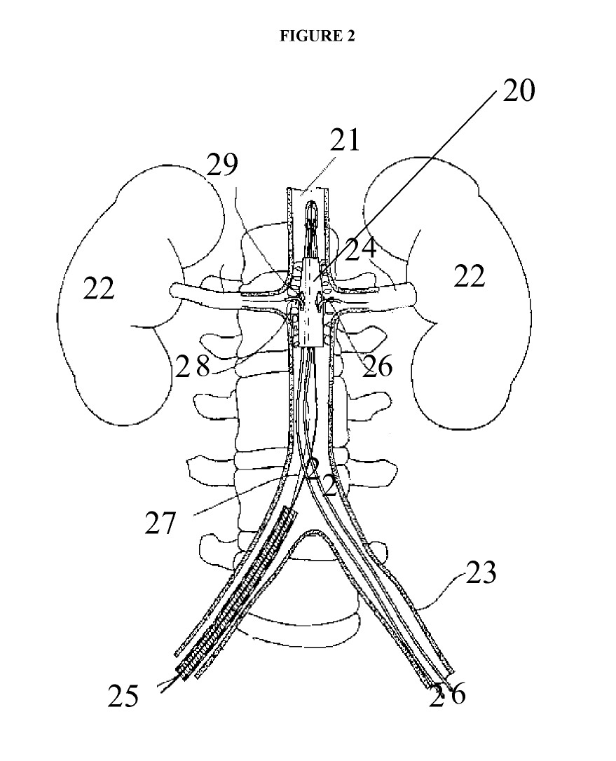

Illustrated in FIG. 2 is a graft 20 within an aorta 21. The graft 20 is separated from the luminal wall of the aorta 21 via a helical catheter 28. The helical catheter 28 is not solid and permits fluid communication within the void or space. The graft 20 is delivered via a left femoral approach 25 and positioned at the level of the renal arteries 24, Fenestration guide wires 26 can be delivered via the other femoral vessel 23 and these can be positioned within the graft 20 in order to perform a fenestration 29 from within either side of the graft. The fenestration in the graft 29 will match the opening of the renal arteries 24 that lead to the kidney 22. The fenestration can be performed through the graft with the fenestration wire 26 and also fenestrate the ostium of the renal artery 24 by the same guide wire 26. For example, fenestration would be useful if a dissection flap is causing malperfusion of the renal artery do to aortic dissection. When the helical catheter 28 is removed the aortic graft 24 will open to its full position via guide wires 27 and oppose the luminal sides of the aorta 21. The fenestrations of the graft 29 will align with the ostium of the renal arteries and permit blood communication through the graft 29 via the renal arteries 24 and thereby allow adequate blood flow to the kidney 22.

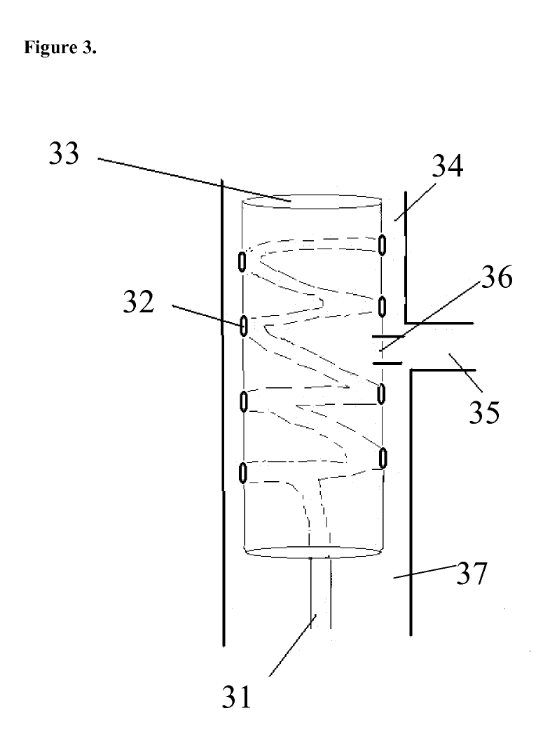

Illustrated in FIG. 3 is a perfusion catheter 31 that is placed inside the aorta 37. The perfusion catheter extends beyond a deployed graft 33 and may be in either the true lumen or false lumen or both. The perfusion catheter will have a number of side holes 32 that permits radiological contrast to enter the luminal space 34 and this will permit imaging of branch vessels 35 in relation to the graft 33. Imaging of the side branches is important as it permits alignment of graft fenestration 36 with a side branch vessel 35. The perfusion catheter can also serve to temporarily anchor the endograft from the wall of the aorta and there by create a space between the graft and the aortic luminal wall. This space permits imaging and also for fenestration to be performed using fenestration instruments.

Illustrated in FIG. 4 is a graft 41 is depicted in situ within the aortic lumen 47. The outer wall of the aorta is defined 46. The position of the fenestration catheter is shown 43 located within the graft 41. The fenestration catheter 43 has a curved tip and the catheter is shown to be stabilized against the graft 41 and or aorta 46 by a stabilizing armature 42A. The fenestration is created in the graft 41 at a position that is directly opposite to the side branch vessel 44 such that there will be blood communication between the lumen of the graft and branch side vessel 44. There is a void or space 45 created between the graft and the aortic wall where the fenestration between graft-aorta and branch vessel is made, this void or space 45 would also permit the placement for an OD perfusion catheter.

Illustrated in FIGS. 4B& 4C is graft and catheter system similar to that of FIG. 4A except that the stabilizing armature 42A is replaced by a balloon 42B.

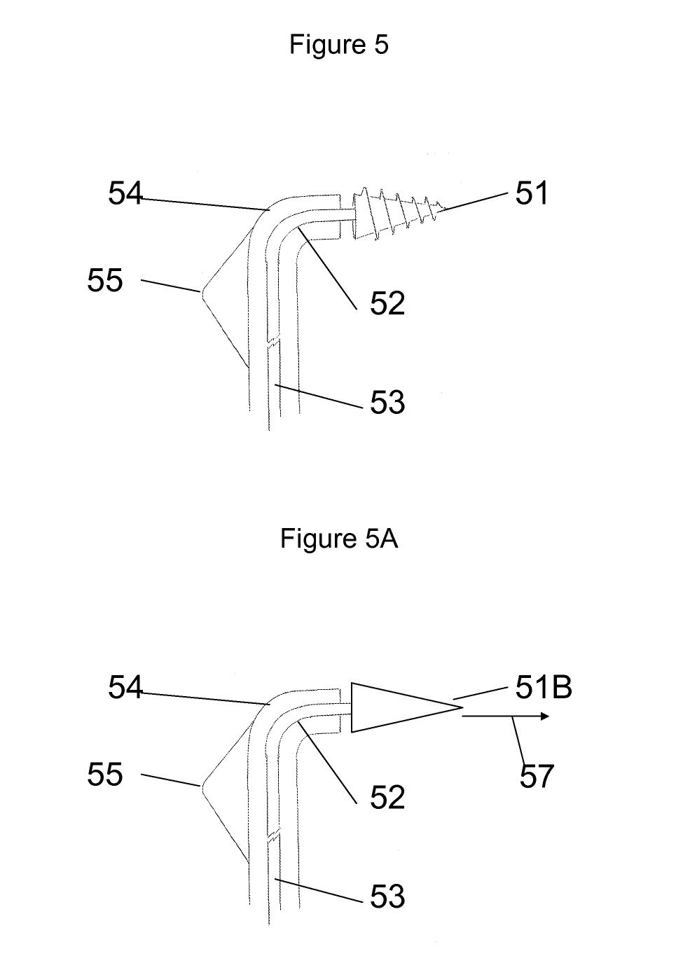

Illustrated in FIG. 5 is a close up view of the distal end of the fenestration catheter. The distal tip of the fenestration catheter has a self tapping fenestration tip 51 that is screw-like. For functional movement of the self tapping fenestration tip 51 the catheter has flexible distal drive shaft 52 and this drive shaft 52 can be driven by a high torque proximal drive shaft 53. The fenestration tip and drive shaft are positioned via the use of an outer guide catheter 54. Once the tip has been located in the correct anatomical position to create a fenestration the catheter is stabilized via the deployment of a guide stabilizer 55 which can be in the form of legs which raise into position as illustrated at item 55, or stabilizing armature or a balloon.

The self tapping tip 51 operates by means of a piercing and opening process where the very sharp pointed tip will pierce a graft or tissue, and once pierced, by rotating the tip by a fraction or whole number of full rotations from the angular starting point, a precise diameter of fenestration can be formed. The diameter is directly proportional to the cone angle and location of the cone and thus the fenestration can be accurately sized.

Illustrated in FIG. 5B is a fenestration tip similar to that of FIG. 5, except that no external helical thread is present on the conical tip 51B. In this case the conical tip 51B can be rotated and will perform the piercing and opening by a combination of translation in the direction of arrow 57 and rotation, as translation continues after piercing has occurred. The diameter of the fenestration will be a function of where the tissue is on the slant height of the conical surface of the tip 51B. The translation can occur by the drive shaft 53 translating with respect to the catheter.

In respect of the embodiment of FIG. 5B, as there is no thread to help keep tissue and or the graft at the desired diametrical opening of the fenestration, locking or location means can be provided in or on the surface of the conical tip 51B, whereby once the desired opening size is achieved, the conical tip and tissue or graft are releasably secured relative to each other, which can assist in stabilization.

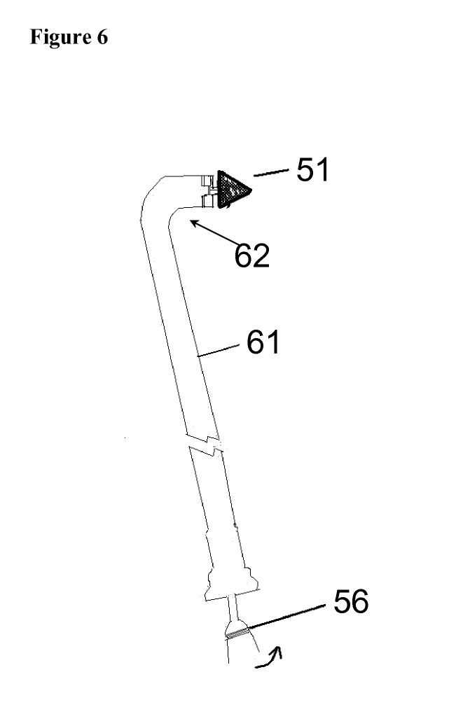

Illustrated in FIG. 6 is the fenestration instrument 50 with the tip of FIG. 5 or 5A that is placed within the aorta and or within an endograft in situ or within the false lumen and or within the true lumen. The instrument includes a catheter 61 with a distal end 62 that consists of a self tapping screw tip 51 or conical tip 51B that is permitted to rotate. The rotation is carried out either by hand or via a mechanical or other motor 56. The self tapping screw permits cutting of tissue or endograft material.

Where ever it is used, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

It will be understood that the invention disclosed and defined herein extends to all alternative combinations of two or more of the individual features mentioned or evident from the text. All of these different combinations constitute various alternative aspects of the invention.

While particular embodiments of this invention have been described, it will be evident to those skilled in the art that the present invention may be embodied in other specific forms without departing from the essential characteristics thereof The present embodiments and examples are therefore to be considered in all respects as illustrative and not restrictive, and all modifications which would be obvious to those skilled in the art are therefore intended to be embraced therein.

1. A method for in situ functional fenestration, said method including the steps of:
positioning a graft or graft unit in situ within a body lumen and
forming an initial void or space between graft unit and the inner wall of said body lumen, and the void or space permits fenestration of the tissue and or graft and closing the void space and allowing the graft, main body lumen and main body side lumen/s to communicate.

2. (canceled)

3. A method as claimed in claim 1, wherein said mechanical fenestration is performed in the graft material.

4. A method as claimed in claim 3, wherein said mechanical fenestration is performed in a tissue associated with a side branch of said body lumen.

5. (canceled)

6. A method as claimed in claim 1, wherein said body lumen is a large blood vessel.

7. A method as claimed in claim 1, wherein said body lumen is an aorta.

8. A method as claimed in claim 7, wherein said void or space is closed or removed after fenestration such that said graft has final apposition with a luminal body wall.

9. A method as claimed in claim 8, wherein said graft fenestration is aligned with a luminal body wall side branch.

10. A method as claimed in claim 1, wherein said graft is in a first open state and then a final greater open state.

11. A method claimed in claim 1, wherein said graft is in a fully open state and held away from the luminal wall to create a void or space.

12. (canceled)

13. A method as claimed in claim 11, wherein a helical catheter is used to create the voice space.

14. A method as claimed in claim 11, wherein the legs on a catheter delivery system for graft delivery are used to create a void or space for fenestration.

15. A method as claimed in claim 1, wherein after fenestration the graft is in communication with inner lumen of the body lumen and side branches connected to the body lumen.

16. A method as claimed in claim 1, wherein a fenestration is created in both the graft and a side branch vessel.

17. A method as claimed in claim 1, wherein there is fluid communication between a side branch vessel and a graft fenestration.

18. A method as claimed in claim 1, wherein said graft is an endograft and or a covered stent and or an open stent metal alloy frame and or an open biomaterial compatible frame.

19. (canceled)

20. A method as claimed in claim 1, wherein said graft is a biocompatible tube.

21. (canceled)

22. (canceled)

23. A method as claimed in claim 18, wherein said stent is covered or coated with a polymeric material.

24. A method as claimed in claim 23 wherein said covering or coating is a polyurethane polycarbonate.

25. (canceled)

26. A method as claimed in claim 3, wherein said fenestration is carried out using one or more than one of the following: surgical laser, harmonic scalpel, guide wire, cutting device, needle, catheter, biopsy apparatus, ablation apparatus, ablation catheter, balloon catheter.

27. A method as claimed in any claim 1, wherein said void or space allows for radiological assessment of side branches of a body lumen for example branches in communication with the aorta.

28. A method as claimed in claim 27, wherein said radiological assessment of said side branches allows for the alignment of the graft fenestration with a side branch ostium.

29. A method as claimed in claim 1, wherein a catheter is used to create a fenestration and the catheter includes a:
a) a distal tip that can perform a piercing and opening action; and or
b) a distal tip rotates by means of a drive shaft within the catheter to cut and or open a tissue or graft to form fenestration; and or
c) a stabilization means to stabilize the position of said catheter during fenestration.

30. A method as claimed in claim 29, wherein said catheter has a distal tip that is conical or helical or screw like to perform a piercing and opening action or a cutting and opening action.

31. A method as claimed in claim 29, wherein said distal tip is self-tapping.

32. A method as claimed in claim 30, wherein said distal tip is drawn into said graft and is self stabilizing.

33. A method as claimed in claim 32 wherein said distal tip or said catheter is stabilized due to the act of fenestration and or completion of fenestration.

34. A method as claimed in claim 32 wherein stabilization of the distal tip or catheter permits accurate alignment of the fenestration with a side branch body lumen and with the side branch vessel originating from a larger body lumen.

35. A method a claimed in claim 32, wherein stabilization of the distal tip or catheter permits alignment of a side branch graft or stent with a side branch vessel in situ through an aortic graft and or stent.

36. A method as claimed in claim 32 wherein stabilization of the distal tip or catheter occurs prior to the act of fenestration via a stabilizing armature and or Balloon.

37. A method as claimed in claim 32 wherein stabilization of said catheter occurs prior to and during the act of fenestration.

38. A method as claimed in claim claim 1, wherein stabilization of the fenestration catheter takes place prior to fenestration via an external stabilizing armature and or balloon.

39. A method as claimed in claim 38, wherein the fenestration catheter includes said armature or balloon.

40. A method as claimed in claim 1, wherein fenestration is made through a branch blood vessel and or an organ and or a aortic dissection flap and or a material related to a graft or stent device.

41. A method as claimed in claim 1, wherein said catheter is self tapping where by it can be drawn the tissue and is self stabilizing.

42-49. (canceled)

50. A method as claimed in claim 1, wherein fenestration is made through smooth muscle and or a graft.

51. A method as claimed in claim 1, wherein the graft fenestration permits biological communication between two lumens.
